(19) United States
(12) Patent Application Publication
(10) Pub. No.: US 2008/0152735 A1
(43) Pub. Date: Jun. 26, 2008

(54) Herbal Extract Having Anti- Influenza Virus Activity and Preparation of Same

(75) Inventors: Wen-Liang Chang, Taipei City (TW); Chen-Wen Yao, Taipei (TW); Ann Chen, Taipei City (TW); Chung-Yang Yen, Taipei (TW); Li-Shian Shi, Huwei Township (TW); Jenn-Han Chen, Taipei City (TW); Ching-Len Liao, Taipei (TW); Kuo-Yuan Hwa, Taipei (TW); An-Rong Lee, Taipei City (TW); Po-Huang Liang, Taipei (TW)

Correspondence Address: WPAT, PC;INTELLECTUAL PROPERTY ATTORNEYS, 2030 MAIN STREET, SUITE 1300, IRVINE, CA, 92614, US

(73) Assignee: NATIONAL DEFENSE MEDICAL CENTER, Taipei (TW)

(21) Appl. No.: 11/963,628

(22) Filed: Dec. 21, 2007

(30) Foreign Application Priority Data
Dec. 25, 2006 (TW) 095148709

(51) Int. Cl. A61K 36/236; A61K 36/00; A61K 36/489; A61K 31/352; A61P 31/12

(52) U.S. Cl. 424/757; 424/725; 514/456

(57) ABSTRACT

The invention provides a herbal extract having anti-influenza virus activity, by extracting Sophora flavescens Ait, Thesium chinense Turcz, licorice, or mixture thereof with solvent. The herbal extract contains at least one compound selected from the group of compounds represented by following formula (I) or (II) or mixture thereof:

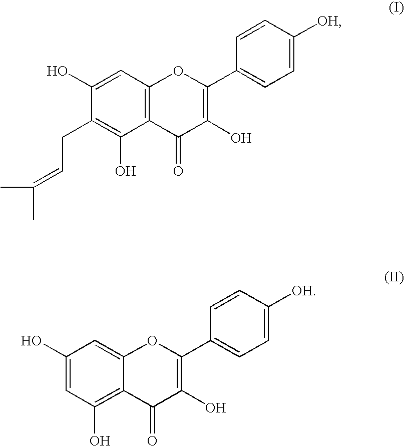

### BACKGROUND OF THE INVENTION

1. Field of the Invention

The invention relates to a herbal extract having anti-influenza virus activity, and in particular to a herbal extract obtained by extracting Sophora flavescens Ait, Thesium chinense Turcz, Licorice or mixture thereof with at least one solvent, wherein an effective component can be obtained after purification of said extract.

2. Description of the Prior Art

Influenza is an infectious disease caused by an influenza virus which is a virus with ribonucleic acid as the genetic material, and is belonged to Orthomyxoviridae on taxonomy of virus. Based on the serum immunological response induced therefrom, influenza virus can be classified into influenza virus A, B or C. Influenza occurs most frequently in winter or early spring. This virus invades into lung generally via mouth and nose. The type of influenza virus varies every year, and areas with crowed population or public places are most susceptible to its infection. Historically, it was known that this virus causes a fever respiratory tract epidemic disease once every 3-4 years, and it also causes a worldwide epidemic infection once every 10-30 years. The 1918 flu pandemic caused at least 2,200 million deaths, and the virus has caused an estimated 60 million deaths worldwide. In addition to death, influenza virus can incur children or senior citizens to be susceptible to serious complications, including nasosinusitis, bronchitis and pneumonia, as well as myocarditis and meningitis concurrent with bacteriemia, and even death due to mindless care. Accordingly, influenza virus has been one of the most important subjects in the world.

Neuraminidase (NA) is a glycoprotein encoded by RNA of the viral genome. Its molecular weight is 50 kD, and it is consisted of 454 amino acids. Each virion has 100 molecules of neuraminidase. The NA of influenza virus A has 9 subtypes (N1˜N9 strains). The hydrophobic region in N-terminal of NA is anchored on the bi-layered lipoid membrane of the virion. The membrane profile assumes mushroom-shaped and is communicated with the hydrophobic region inside the membrane via a short rod part. The homologies among the head sequences of NA proteins of N1˜N9 strains of influenza virus A are about 42%˜57%. Since the number and position of cysteine residues within each type and subtype of NAs are close with one another, it is suggested that their three-dimensional structures are very similar. NA is present as a homotetramer, i.e. constructed through binding of four NA monomers. The head of the monomer is consisted of 6 helical β-pleated sheets. Its basal binding site (binding with neuraminic acid) is positioned at its head. It is present as a pocket-like shape and is highly conserved. NA is modified via glycosylation by Golgi bodies. The functions of NA comprise of hydrolyzing N-acetylneuraminic acid at termini of various polysaccharide receptors on cell membrane, promoting the release of progeny viruses from cell membrane, preventing the aggregation of virions, assisting the penetration of virions through the mucus of the respiratory tract, as well as facilitating the spreading and adsorption of virions on the susceptible cell. Clinical application of NA inhibitors, Oseltamivir and Zanamivir, result in their specific binding with the segment of conserved residues within the active site of influenza viral NA, which induces the inactivation of the enzyme, blocks the cleavage of terminal neuraminic acids attached on glycoprotein and glycolipid, and ultimately, results in the inability of the influenza virion to detach from the surface of the host cell such that the virus is prevented from spreading over the mucosa of the respiratory tract.

Sophora flavescens Ait is the root of a perennial plant of Leguminosae, which has effects of alleviating fever, de-rheumatism, repelling chill, killing worm, diuresis and the like, and hence it can be used to treat hot dysentery, hematochezia, jaundice, anuresis, red leucorrhea, genitalia swollen and itching, eczema, wet boil, skin pruritus, scabies, and leprosy, as well as to treat locally trichomonad vaginitis. (Pharmacopeia, People Republic of China, A COLOURED ATLAS OF THE CHINESE MATERIA MEDICA SPECIFIED, 1995, p. 263).

Chemical ingredients in Sophora flavescens Ait comprise essentially alkaloids and flavones. Flavone ingredients in Sophora flavescens Ait have been known heretofore to exhibit following pharmacological activities: 1. anti-diabetes and cataract complications thereof; 2. inhibiting cAMP phosphodiesterase; 3. inhibiting phosphatase Cg1 (PLCg1); 4. anti-cancer; 5. anti-inflammatory action; 6. anti-pathogen, and inhibiting fungus, Gram positive bacteria, Staphylococcus auresus, Streptococcus, and vagina trichomonad; 7. anti-arrhythmia; and the like (Huichuan, Zhao and Wen Ji, Sun, Flavonoid Ingredients in Sophora flavescens Ait and Pharmocological Study Thereof, Traditional Chinese Materials, 2005, 28(3): 247-251). Total matrine has activities of anti-Coxsackie virus B (CVB) and anti-Hepatitis B virus (Zhang, H. L. et al., Advances in the research on bioactivity of Sophora flavescens, J. Northwest Sci-Tech Univ. Agri and For., 2004, 32(5): 31-37.).

Pharmacologically, Sophora flavescens Ait can slow heart rate, weaken the contraction force of cardiac muscle, and reduce cardiac output. All of the Sophora flavescens Ait, matrine, and flavone have anti-arrhythmia action. Sophora flavescens Ait injection has quicker and persistent action against arrhythmia induced by aconitine, and it also has anti-hypertension activity. The deconction of Sophora flavescens Ait exhibits inhibitory action against Bacillus tubercle, Shigella dysenteriae, Staphylococcus auresus, and Escherichia coli, as well as against a number of skin fungus. In addition, it demonstrates activities such as, diuresis, anti-inflammatory, anti-allergy, sedation, and relieving asthma, eliminating sputum, increasing white blood cell, anti-tumor and the like.

Thesium chinense Turcz is a perennial herbal plant of Santalaceae. It is produced in various places in Mainland China and Taiwan, and it wildly grows at roadside of brae or field. The whole plant of Thesium chinense Turcz can be used as drug, and it has effects of alleviating fever, disintoxicating, invigorating the kidney, holding sperm, and anti-inflammatory. The whole plant of Thesium chinense Turcz has been used in traditional medicine for treating acute cystitis and head boil, lymphatic tuberculosis, as well as treating bone fracture, acute mastitis, pharyngolaryngitis, pneumonia, upper respiratory tract infection, kidney empty, lumbago, wet dream, holding sperm and the like. Thesium chinense Turcz contains chemical ingredients such as flavones, organic acids, alkaloid, phenolics and volatile oils, with flavones such as kaempferol present at higher content. It has been used in Mainland China to produce pharmaceutical dosage form, for example, Thesium chinense Turcz tablet, granules, syrup and the like. (Li Yuchih, Isolation and Identification of Kaempferol from Thesium chinense Turcz, Journal of Traditional Chinese Medicine, 2001, 15(3):23).

Licorice is the dried root or rhizome of Leguminosae plants such as Glycyrrhiza glabra Lin., Spanish licorice, G. Inflata Bat., or G. uralensis L. Ingredients in licorice comprise more than 100 types of compounds which primarily include triterpenoids, flavonoids, coumarins, lignans, alkaloids, organic acids, sugars, licoflavonols, and the like. Licorice has effects of replenishing the spleen, profiting airy energy, smoothing the lung, relieving a cough, slowing emergency, analgesia, alleviating fever, disintoxicating and the like.

Since influenza virus infection might result in pathological change of cells, preventing and treating methods currently employed include vaccine and anti-virus agents. Vaccine prevention approach may be effective, but it is not applied generally. In the aspect of anti-virus agents, no significant breakthrough has been revealed. Therefore, it is of great emergency to develop an extract from Chinese herbal medicine for inhibiting effectively the influenza virus. It will be of significant benefit for clinical treatment of influenza virus if Sophora flavescens Ait, Thesium chinense Turcz, and licorice can be applied on the inhibition or prevention of influenza virus.

### SUMMARY OF THE INVENTION

In view of lack of effective conventional drug for treating influenza virus, one of objects of the invention is therefore to provide herbal extract having anti-influenza virus activity and with its main goal for preventing, inhibiting or killing influenza virus.

Another object of the invention is to provide a method for suppressing influenza virus ex vivo by using extract from Sophora flavescens Ait, Thesium chinense Turcz, and licorice.

Yet another object of the invention is to provide a pharmaceutical composition having anti-influenza virus activity, comprising extract from Sophora flavescens Ait, Thesium chinense Turcz, licorice, or their rhizome mixture, which contains chemical substance having anti-influenza virus activity.

In order to achieve above-mentioned objects, the invention provides a herbal extract having anti-influenza virus activity, which can be obtained by extracting Sophora flavescens Ait, Thesium chinense Turcz, licorice, or their rhizome mixture with solvent. The suitable solvents used in the invention include alcohol, methanol, acetone, ethyl acetate, water, or mixture thereof. The inventive extraction comprises compound represented by following formula (I) or (II), or mixture thereof:

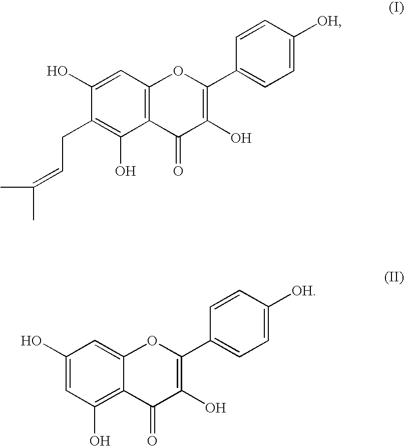

In a preferred embodiment, compound of formula (I) can be prepared by a process consisting of following steps: (a) extracting roots of Sophora flavescens Ait or licorice with acetone and methanol to obtain a crude extract containing acetone and methanol; (b) dissolving the crude extract from step (a) in water, and then extracting the aqueous solution with ethyl acetate to obtain an ethyl acetate layer; (c) purifying the ethyl acetate layer from step (b) to obtain compound (I). Preferably, the purification in step (c) is achieved by means of filtration or silica gel chromatography.

In a preferred embodiment, compound of formula (II) can be prepared by a process consisting of following steps: (a) extracting Thesium chinense Turcz with alcohol to obtain an extraction solution; (b) concentrating the extraction solution from step (a), and then hydrolyzing the concentrate to give a hydrolyzing solution; (c) extracting the hydrolyzing solution from step (b) with ethyl acetate to obtain an ethyl acetate extraction solution; (d) purifying the ethyl acetate extraction solution from step (c) to obtain compound (II). Preferably, the concentration in step (b) and the purification in step (d) are achieved by means of chromatography.

The invention also provides a pharmaceutical composition which comprises effective amount of the above-mentioned compound of formula (I) or (II) and pharmaceutically acceptable carriers or diluents which are used together with the compound. The pharmaceutical composition can be used for preventing, treating, or killing influenza virus.

Moreover, the invention provides a method for suppressing influenza virus activity in vitro by contacting the above-mentioned herbal extract or the pharmaceutical composition with influenza virus to thereby inhibit the activity of the virus.

Accordingly, the invention takes advantage of extract from Sophora flavescens Ait, Thesium chinense Turcz, licorice, or mixture thereof and a pharmaceutical composition containing compounds of formula (I) and/or formula (II) to inhibit effectively the activity of influenza virus. Therefore, the extract and the pharmaceutical composition can be used clinically for preventing or treating influenza.

These features and advantages of the present invention will be fully understood and appreciated from the following detailed description of the accompanying Drawings.

### BRIEF DESCRIPTION OF THE DRAWINGS

None.

### DETAILED DESCRIPTION OF THE PREFERRED EMBODIMENT

The invention provides extracts containing licoflavonol or kaempferol from Sophora flavescens Ait, Thesium chinense Turcz, licorice, or mixture thereof, and it also provides a pharmaceutical composition containing compounds of formula (I) and/or (II), for treating or preventing influenza virus.

In order to achieve better extraction effect, prior to the extraction step according to the invention, the Chinese herbal medicine materials described above can be subjected to physical means such as stamping, grinding, chopping, and the like, to make them into small particles as far as possible. Preferably, it is carried out by grinding, and more preferably, one or more Chinese herbal medicine materials described above are ground into powder-like materials in order to facilitate further extraction.

After extracting Chinese herbal medicine materials described above with solvents, the extract can be used to formulate into products used in various applications. However, in order to increase the purity of effective ingredients in the extract, various purification steps can be performed as desired after the extraction steps according to the invention. Methods suitable for the purification of the extract are not necessary to teach specifically, and are well known to one skilled in the art. Purification methods that can be used in the invention include such as, but not limited to, chromatography, crystallization, filtration, precipitation and the like, depending on the purpose desired to be achieved.

Herbal extracts having anti-virus activity prepared according to the invention can be used as purified or unpurified forms, or preferably, used together with carriers, diluents, excipients or adjuvant conventionally used in formulation art. To this end, they can be formulated as appropriate by conventional manner into emulsifiable concentrate (for example, as lotion, detergent, washing concentrate, shampoo, and the like), coating paste (for example, as coatings), direct spraying solutions (for example, as sprays), diluted solution (for example, as beverage, healthy foods), components as filling material (for example, in toys, swabs), powder to be mixed with carriers, soluble powder, dusts, granules. Moreover, they can be encapsulated, for example, to be used in air filter, water filtering material, mouthpiece content, or filtering membrane. As the type of the formulation, it can be selected based on the desired purposes and the main environmental condition, such as application method including spray, atomizing, dusting, spreading, coating, or emulsifying. The formulation can contain additionally adjuvant, such as, stabilizer, antifoam, viscosity modifier, binder, or thickener, as well as other additives used in formulation having special effect.

Optionally, the herbal extract having anti-virus activity prepared according to the invention can be formulated into a pharmaceutical composition for treating or prevention influenza virus infections. The herbal extract having anti-virus activity prepared according to the invention can be used alone or in combination with pharmaceutically acceptable carriers or excipients, and it also can be administrated as single dosage form or multiple dosage form. Pharmaceutically acceptable carriers or diluents and other adjuvants or excipients known in the art can be formulated according to conventional techniques, for example, referring to Remington's Pharmaceutical Sciences, 19^{th} edition, Gennaro ed., Mack Publishing Company, Easton, Pa. (1995).

Pharmaceutical compositions can be formulated specifically for using in any suitable administrating routes, such as, oral, rectal, nasal, lung, topical (including buccal, sublingual), transdermal, intracisterna, intraperitoneal, vaginal, and parenteral (including subcutaneous, intramuscular, intraspinal, intravenous, and intracutaneiou). It should be appreciated that a preferable administration route is determined by the general symptom, age of the subject to be treated, characteristics of the symptom to be treated, and the active ingredients selected.

The pharmaceutical compositions used in oral administration may include solid dosage form, such as capsule, tablet, dragée, pill, powder, and granule. Suitably, they can be prepared with film coated (such as enteral film) by methods well known to the person skilled in the art. Alternatively, they can be formulated as for controlled release of the active ingredient, such as, sustained- or extended-release.

Liquid dosage forms used in oral administration include solution, emulsion, suspension, syrup, and elixir.

Pharmaceutical composition used in parenteral administration includes sterile aqueous and non-aqueous injection solutions, dispersion, suspension or emulsion, as well as sterile powder to be reconstituted in sterile injection solution or dispersion before use.

Other suitable administration forms include suppository, spray, ointment, cream, gel, inhalant, patch, implant, and the like.

The actual dosage of the herbal extract having anti-influenza virus activity prepared according to the invention is determined by factors such as the frequency and mode of administration, sex, age, body weight, and general condition of the subject to be treated, the character and seriousness of the symptom to be treated as well as any associated disease.

The technique of feature of the invention will be illustrated in more detail by way of the following examples which do not limit the scope of the invention. Various variations and modifications can be made by one skilled in the art without departing from the spirit and scope of the invention.

### EXAMPLES

### Example 1 Preparation of Extract from Sophora flavescens Ait

5.4 kg Sophora flavescens Ait was extracted with acetone and methanol respectively to give a crude acetone extract (94.5 g) and a crude methanol extract (218 g). The crude methanol extract was dissolved in water, extracted with ethyl acetate (EtOAc) to yield an ethyl acetate layer (72.0 g) and an aqueous layer (135.8 g). The ethyl acetate layer was isolated and purified by silica gel chromatograph to yield 20.0 mg licoflavonol (SOFL-EM-8-6C), which was further re-crystallized from methanol to get yellow crystal.

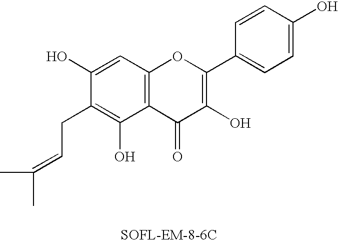

The chemical properties of Licoflavonol (SOFL-EM-8-6C) were as follow: MP 185˜187° C. (decomp.); UVλmax EtOH (log ε): 256 (4.05), 271 (4.15), 295 sh (3.86), 304 sh (3.87), 340 inf. (4.06), 369 (4.13) nm.

¹H-NMR (300 MHz, CD₃OD) δ 1.68 (3H, s), 1.81 (3H, s), 3.51 (2H, br d), 5.22 (1H, m), 6.24 (1H, s), 6.91 (2H, d, J=8.8 Hz), 8.10 (2H, d, J=8.8 Hz).

¹³C-NMR (125 MHz, Me₂CO-d₆) δ 17.9 (C-4″), 21.9 (C-1″), 25.8 (C-5″), 93.9 (C-8), 104.1 (C-10), 111.7 (C-6), 116.3 (C-3′, 5′), 123.2 (C-2″), 123.4 (C-1′), 130.4 (C-2′, 6′), 131.6 (C-3″), 136.5 (C-3), 146.7 (C-2), 155.6 (C-9), 158.9 (C-5), 160.1 (C-4′), 162.6 (C-7), 176.5 (C-4).

EIMS (30 eV): m/z 354 ([M]+, 100%), 311 (80%), 299 (75%), 121 (25%).

### Example 2 Preparation of Extract from Thesium chinense Turcz

1.6 kg Thesium chinense Turcz was extracted three times each with 70% alcohol under reflux for 3 hours. Extracts were combined and concentrated, passed through a macroporous resin chromatographic column, which was eluted first with water and then with 70% alcohol. The eluate was concentrated and then was added with 5% sulfuric acid/dilute alcohol solution, which was hydrolyzed in a boiling water bath for 3 hours. The hydrolyzed solution was extracted 6 times with ethyl acetate. The extracts was combined and washed with water until neutralized. The obtained solution was subjected to polyamide chromatography to get 6.5 g kaempferol (SOFL-EM-8-6S).

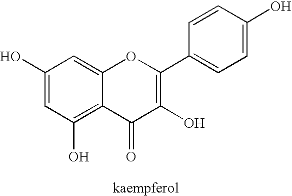

Chemical properties of Kaempferol (SOFL-EM-8-6S) were as follow: MP 277˜279° C.; UVλmax MeOH (log ε): 267 (4.25), 367 (4.15) nm.

¹H-NMR (300 MHz, DMSO-d₆) δ 6.16 (1H, d, J=2.1 Hz), 6.37 (1H, d, J=2.1 Hz), 6.89 (2H, d, J=8.7 Hz), 8.05 (2H, d, J=8.7 Hz).

¹³C-NMR (125 MHz, DMSO-d₆) δ 93.4 (C-8), 98.3 (C-6), 102.7 (C-10), 115.3 (C-3′, 5′), 121.6 (C-1′), 129.4 (C-2′, 6′), 135.4 (C-3), 146.6 (C-2), 156.0 (C-9), 158.6 (C-4′), 160.5 (C-5), 163.5 (C-7), 175.8 (C-4).

### Example 3 Anti-Influenza Virus Test Performed with Sophora flavescens Ait or Thesium chinense Turcz Extracts of the Invention

Sophora flavescens Ait extract form example 1 and Thesium chinense Turcz extract from example 2 were subjected to anti-influenza virus test in vitro according to the following test process:
1. Culturing MDCK cells (Madin-Darby Canine Kidney Epithelial Cells) first on one day before drug selection in a 96-well cell culturing plate by inoculating 2*10⁴ cells in each well.
2. After culturing for 18˜24 hours, aspirating off cell culturing medium, washing with DPBS twice, and finally, inoculating 100 μl/well of virus culture, then storing in a cultivation box till processing with test drug later.
3. Dilution of test drug:
   a. Diluting drug into 1 mg/ml:
      Crude extract (original concentration 200 mg/ml in DMSO): in each well of a 96-well cell culturing plate, 1 μl of test drug was added into 199 μl of virus culture.
      Purified ingredient (original concentration 50 mg/ml in DMSO): in each well of a 96-well cell culturing plate, 1 μl of test drug was added into 49 μl of virus culture.
   b. Preparation of crude extract into solutions of two concentrations of 300 μg/mL and 100 μg/mL:
      300 μg/ml: Adding 90 μl of 1 mg/ml of drug into 210 μl of virus culture.
      100 μg/ml: Adding 30 μl of 1 mg/ml of drug into 270 μl of virus culture.
   c. Preparation of purified ingredients into solutions of two concentrations of 100 μg/mL and 20 μg/mL:
      100 μg/ml: Adding 30 μl of 1 mg/ml of drug into 270 μl of TPCK culture medium.
      20 μg/ml: Adding 6 μl of 1 mg/ml of drug into 294 μl of TPCK culture medium.
4. Application concentration of test drug:
   Crude extract: Two concentrations were applied: 150 μg/ml and 50 μg/ml.
   Purified ingredients: Two concentrations were applied: 50 μg/ml and 10 μg/ml.
5. Infection dosage of influenza virus (H1N1): Inoculating 50 μl/well of 200 PFU (plaque forming unit) of influenza virus, this dosage of virus corresponding to a condition of 100-300 TCID₅₀.
6. Processing: in two conditions:
   a. Processing with test drug alone (D): Removing original virus culturing medium, adding 50 μl of diluted test drug, and adding fresh virus culturing medium after one 1 hour.
   b. Inoculating infectious virus (D1h+V) one hour before processing with test drug: Aspirating original virus culturing medium, adding 50 μl of diluted test drug, and adding 50 μl of fresh virus culturing medium after one 1 hour.
      In each plate, one row of wells (without inoculating cells) was used for blank group, one row of wells for virus control group, and one row of wells for Mock control group.
7. Reading of Results:
   After two or three days, when the apoptosis rate of cells in virus control group observed under microscope is more than 75%, MTT test was performed:
   a. Compared with mock control group, absorbance percentage≦25 was recorded as +/−; >25≦50 recorded as +; >50≦75 as ++; >75≦100 as +++; >100 as ++++
   b. The reading of virus control group had to be ≦50, it is considered to be effective when the apoptosis caused by virus was recovered by test drug to higher than +++.

A test drug was estimated as an effective drug when it was effective in two repeated experiments. The experimental results are shown in Table 1.

In an in vitro anti-influenza virus test, SOFL-EM-8-6C fraction in Sophora flavescens Ait extract exhibited the most strong anti-influenza virus activity. When infecting with virus (D1+V) at one hour after pretreatment with test drug, cell survival rate could be up to 100% with 10 μg/mL of SOFL-EM-8-6C fraction, this indicated that this Sophora flavescens Ait extract fraction had actions for preventing the infection of influenza virus or inhibiting influenza virus. Upon addition of test drug (V1+D) at one hour subsequent to adsorption of virus on cell, 10 μg/mL of SOFL-EM-8-6C fraction could result in cell survival rate of 100%, indicated that the Sophora flavescens Ait extract fraction could inhibit effectively the activity of influenza virus. While treating cell simultaneously (DV) with test drug and virus, 10 μg/mL of SOFL-EM-8-6C fraction could result in 75% of cell survival rate, indicated that the Sophora flavescens Ait extract fraction could inhibit instantaneously the apoptosis induced by influenza virus.

It can be seen from Table 1 that the SOFL-EM-8-6S fraction obtained from Thesium chinense Turcz also has the activity of inhibiting influenza virus. When infecting with virus (D1+V) at one hour after pretreatment with test drug, cell survival rate could be up to 75% with 18.75 μg/mL of SOFL-EM-8-6S fraction. This indicates that the Thesium chinense Turcz extract fraction had actions for preventing the infection of influenza virus or inhibiting influenza virus. According to analysis, the Sophora flavescens Ait extract contains a compound represented by formula (I), and the SOFL-EM-8-6S fraction of Thesium chinense Turcz contains a compound of formula (II):

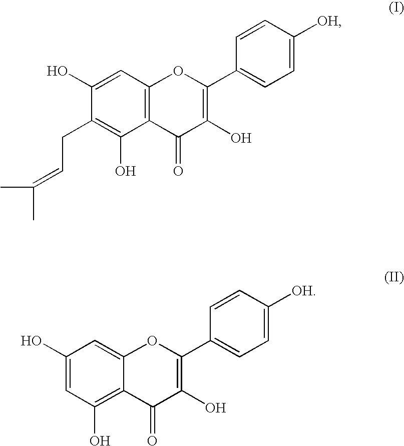

Furthermore, according to analysis, licorice also contains the compound of formula (I); thus, it could be suggested that licorice also has the anti-influenza virus activity like that of Sophora flavescens Ait extract.

These compounds were tested for the inhibitory activity against neuraminidase (NA) of influenza virus. Neuraminidase was expressed in ovary cell of Chinese hamster as following:

Chinese hamster ovary (CHO) cell (BCRC 60006, purchased from the Bioresource Collection and Research Center of Food Industry Research and Development Institute (FIRDI) was cultured in a 90% Ham's F-12 medium supplemented with 10% Fetal bovine serum (Gibco Com.), replacing the culturing medium once every three days. Transfection was performed in a 24-well plate (NUNC Com.) by inoculating in each well with 2×10⁵ cells/500 μL into antibiotics-free culturing medium. After pre-culturing for about 16 hours, transfection was carried out. 1 μg green fluorescence plasmid (pEGFP N2) carrying target gene was mixed with culturing medium containing no serum, and the volume of the mixture was adjusted to 50 μL. Separately, lipofectamine 2000 reagent (Invitrogen com.) in a weight three times that of the plasmid was mixed with culturing medium containing no serum, and the volume of the mixture was adjusted also to 50 μL. Immediately after the reaction of this mixture at room temperature for 5 minutes, it was mixed homogeneously with the plasmid-culturing medium mixture prepared above. This 100 μL mixture was allowed to react at room temperature for 20 minutes, and then it was inoculated in a 24-well plate containing normally growing cells. The plate was shaken slightly to enable the plasmid-lipofectamine 2000 mixture to contact with cells more, and therefore, it results in a better transfectiviy. The plate was incubated in a incubator (NUAIRE Com.) at 37° C. and 5% CO₂ for 4˜6 hours, and then replacing the medium with fresh culturing medium. Plasmid bearing green fluorescence emitted light after 16 hours, which was utilized to carry out single cell colony selection, wherein the microscope used was a Nikon ECLIPSE TE2000-U system. The excitation wavelength of the green fluorescence was 488 nm, and its absorption wavelength was 508 nm.

At this time, culturing medium was adjusted to contain 400 μg/mL of G418 as the selection target, and the cell count was adjusted to be 1×10⁴/mL. 5 μL cell suspension was mixed with 10 ml culturing medium, and the resulting mixture was injected via a 12-channel dispenser into a 96-well plate, 100 μL for each well. Then, 0.5 cell/well was inoculated in the plate, and the plate was incubated in an incubator. After about 5 days, single cell colonies were merged. The most intense green fluorescence colony was picked, and its expression and amplification was continued. As the cell count became sufficient, the cell was collected and the membrane protein was isolated for enzymatic activity test.

Membrane protein isolation was carried out with Mem-PER Eukaryotic Membrane Protein Extraction Reagent Kit from PIERCE. Cells were washed with ice-cold PBS and collected by scraping with a cell scraper into a centrifuge tube. Centrifugation was carried out at 1,000 rpm for 2 minutes, and the supernatant was discarded. 150 μL reagent A (containing protease inhibitor) was added into each of 5×10⁶ cells, and reacted at room temperature for 10 minutes. Thereafter, a solution containing 450 μL reagent B plus reagent C (1:2) was added and reacted on ice for 30 minutes under vibrating once every 5 minutes. Subsequently, the reaction mixture was centrifuged at 4° C. (10,000×g) for 3 minutes. The supernatant was transferred into a new centrifuge tube, and reacted at 37° C. for 10˜20 minutes. Then, the cell membrane was separated from cytoplasm, and centrifuged at room temperature (10,000×g) for 2 minutes. The membrane protein layer at the bottom of the tube was collected, and it was subjected to enzymatic activity test using Amplex Red Neuraminidase (Sialidase) Assay Kit from Molecular Probes. At first, a stock solution was prepared. Materials used in the kit included: Component A: Amplex Red reagent; Component B: DMSO; Component C: horseradish peroxidase; Component E: 5× Reaction Buffer; Component F: galactose oxidase; Component G: fetuin. 100 μL Component B was added into Component A to form 10 mM Amplex Red reagent. The 5× Reaction Buffer was diluted with distilled water and used as Reaction Buffer. 200 μL Reaction Buffer was added into Component C to prepare 100 U/mL HRP. 500 μL Reaction Buffer was added into Component F to prepare 200 U/mL galactose peroxidase. Finally, 1.2 mL of Reaction Buffer was added into Component G to prepare 10 mg/mL fetuin. After completion of the preparation of the above-mentioned solutions, solutions were mixed successively in accordance with the following ratio to prepare 100 μM Amplex Red reagent as 2× working solution, which containing 0.2 U/mL HRP, 4 U/mL galactose oxidase, and 500 μg/mL fetuin. 500 μg/mL fetuin was made by adding 50 μL of 10 mM Amplex Red reagent, 10 μL of 100 U/mL HRP, 100 μL of 200 U/mL galactose peroxidase, and 250 μL of 10 mg/mL fetuin into 4.59 mL of Reaction Buffer with a final volume of 5 mL.

After preparation of 2× Amplex Red reagent working solution, the enzymatic activity of neuraminidase was measured as described below: adding 12 μL of Reaction Buffer, and then adding 1 μL of neuraminidase into a 384-well dark plate (PerkinElmer), and finally, adding 12 μL of 2× working solution of the Amplex Red reagent in the plate. The resulting mixture was homogenized, and then the plate was incubated in a oven at 37° C. for 15 minutes. The enzymatic kinetics was measured with Fluoroskan Ascent fluorescence plate detector (Thermo). The assay principle of this kit relied on the cleavage of sialic acid from fetuin by the action of neuraminidase to form desialiated galactose. Under the action of HRP, H₂O₂ generated through oxidation of this desialiated galactose by galactose oxidase and Amplex Red reagent were reacted to form a red fluorescent product-resorufin. With an excitation light of 485 nm, resorufin would emit fluorescent light at 590 nm. A change of fluorescent emission throughout this reaction was detected with a fluorescence plate detector once every 30 seconds. 10 points were taken over 5 minutes. Data were transformed into a slope plot by using Ascent Software of Fluoroskan Ascent as an enzymatic activity curve. Since the assay was a continuous reaction, measurement of change in emitting light at multiple points could reflect its enzymatic reaction kinetics.

The results are shown in Table 2. With same dosage (0.5 mg/mL), the activity of the SOFL-EM-8-6C fraction (0.02 FU/min) was two-fold of that of current anti-influenza virus drug, Tamiflu (0.01 FU/min), while the activity of SOFL-EM-8-6S fraction (0.06 FU/min) was six-fold of that of Tamiflu. This indicated that extracts from Sophora flavescens Ait and Thesium chinense Turcz had a superior inhibitory effect on neuraminidase of influenza virus than that of current commercial available drugs.

TABLE 1
Comparison on Anti-influenza virus activity of Ribavirin
and extract fractions according to the invention

| Medicament | Dosage (μg/mL) | D1 + V | V1 + D | DV | D |
|---|---|---|---|---|---|
| Ribavirin | 9.37 | +++ | | | ++++ |
| SOFL-EM-8-6C | 10 | ++++ | ++++ | +++ | ++++ |
| SOFL-EM-8-6S | 18.75 | +++ | | | ++++ |

TABLE 2
Assay on activity against neuraminidase of influenza virus

| Medicament | Dosage | FU/min | Response |
|---|---|---|---|
| Kit (positive control) | | 0.42 | |
| Tamiflu | 0.5 mg/mL | 0.01 | Inhibition |
| SOFL-EM-8-6C | 0.5 mg/mL | 0.02 | Inhibition |
| SOFL-EM-8-6C | 0.05 mg/mL | 0.13 | Inhibition |
| SOFL-EM-8-6S | 0.5 mg/mL | 0.06 | Inhibition |

In summary, it is apparent from experimental results described above that herbal extract prepared according to the invention can combat influenza virus effectively. Therefore, pharmaceutical composition containing extracts that are prepared from Sophora flavescens Ait, Thesium chinense Turcz, licorice, or mixture thereof according to the invention and comprise licoflavonol or kaempferol can be used clinically to prevent or treat influenza. Furthermore, extracts from Sophora flavescens Ait, Thesium chinense Turcz, licorice, or mixture thereof prepared according to the invention can be applied in air filter, filtering membrane, mouthpiece, lotion, water filtering material, coating material, swabs and the like, which can adsorb virus thereon. Thereby, virus can be isolated on the material described above to prevent human body form contacting with and thus infected by virus. Moreover, materials possessing anti-virus activity can inactivate those viruses adhered thereon to eliminate the infectivity of those viruses. Therefore, it can preclude the propagation route of virus and facilitate greatly the control on viral spread.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. A herbal extract having anti-influenza virus activity, obtained by extracting Sophora flavescens Ait, Thesium chinense Turcz, licorice, or mixture thereof with a solvent.

2. A herbal extract as recited in claim 1, wherein the solvent is one selected from the group consisting of alcohol, methanol, acetone, ethyl acetate, water, or mixture thereof.

3. A herbal extract as recited in claim 1, wherein the extracts contain compounds of formula (I), formula (II), or mixture thereof:

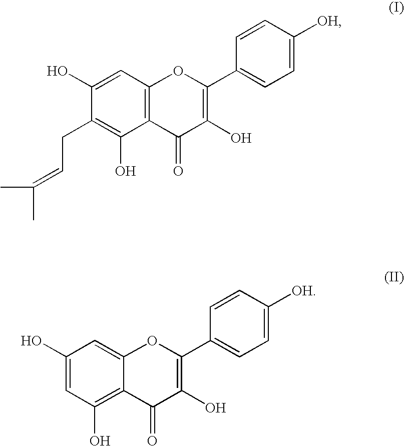

4. A herbal extract as recited in claim 3, wherein the compound of formula (I) is prepared by a process comprising following steps:
(a) extracting roots of Sophora flavescens Ait or licorice with acetone or methanol to obtain acetone extract and methanol extract;
(b) dissolving the methanol extract from step (a) in water, and extracting the resulting solution with ethyl acetate to obtain an ethyl acetate extract; and
(c) purifying the ethyl acetate extract from step (b) to obtain compound (I).

5. A herbal extract as recited in claim 4, wherein the purifying in step (c) is accomplished by filtration or silica gel chromatography.

6. A herbal extract as recited in claim 3, wherein the compound (II) is prepared by a process comprising following steps:
(a) extracting Thesium chinense Turcz with alcohol to obtain an alcohol extracting solution;
(b) concentrating the alcohol extracting solution from step (a), and hydrolyzing the resulting concentrate to obtain a hydrolyzing solution;
(c) extracting the hydrolyzing solution from step (b) with ethyl acetate to obtain ethyl acetate extracting solution; and
(d) purifying the ethyl acetate extracting solution from step (c) to obtain compound (II).

7. A herbal extract as recited in claim 6, wherein the concentration in step (b) and the purifying in step (d) are accomplished by chromatography.

8. A pharmaceutical composition using for preventing, treating, or killing influenza virus, comprises an effective amount of compounds of formula (I) or formula (II) showing as follow: 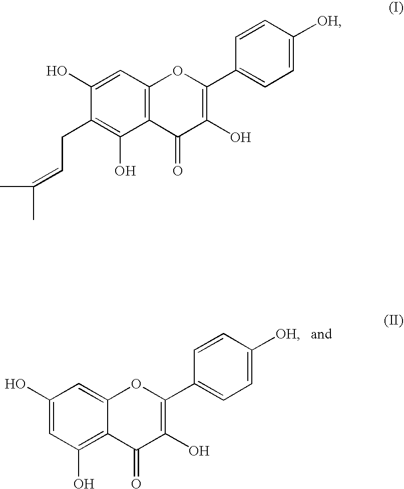
pharmaceutically acceptable carriers or diluents which are used together with the compound.

9. A pharmaceutical composition as recited in claim 8, wherein the compounds of formula (I) or (II) are obtained by using Sophora flavescens Ait, Thesium chinense Turcz, licorice, or mixture thereof as the extraction sources.

10. A pharmaceutical composition as recited in claim 8, wherein the compounds of formula (I) or (II) are obtained by extracting Sophora flavescens Ait, Thesium chinense Turcz, licorice, or mixture thereof with at least one solvent, and then isolating and purifying the resulting extract.

11. A pharmaceutical composition as recited in claim 10, wherein the solvent is one selected from the group consisting of alcohol, methanol, acetone, ethyl acetate, water, or mixture thereof.

12. A pharmaceutical composition as recited in claim 8, wherein the compound of formula (I) is prepared by a process comprising following steps:
(a) extracting roots of Sophora flavescens Ait or licorice with acetone or methanol to obtain acetone extract and methanol extract;
(b) dissolving the methanol extract from step (a) in water, and extracting the resulting solution with ethyl acetate to obtain an ethyl acetate extract; and
(c) purifying the ethyl acetate extract from step (b) to obtain compound (I).

13. A pharmaceutical composition as recited in claim 12, wherein the purifying in step (c) is accomplished by filtration or silica gel chromatography.

14. A pharmaceutical composition as recited in claim 8, wherein the compound (II) is prepared by a process comprising following steps:
(a) extracting Thesium chinense Turcz with alcohol to obtain an alcohol extracting solution;
(b) concentrating the alcohol extracting solution from step (a), and hydrolyzing the resulting concentrate to obtain a hydrolyzing solution;
(c) extracting the hydrolyzing solution from step (b) with ethyl acetate to obtain ethyl acetate extracting solution; and
(d) purifying the ethyl acetate extracting solution from step (c) to obtain compound (II).

15. A pharmaceutical composition as recited in claim 14, wherein the purifying in step (c) is accomplished by chromatography.

16. A method for suppressing influenza virus in vitro, comprising contacting a herbal extract as recited in any one of claim 1 to 7 with the virus, thereby inhibiting activity of said virus.

17. A method for suppressing influenza virus in vitro, comprising contacting a pharmaceutical composition as recited in any one of claim 8 to 15 with the virus, thereby inhibiting activity of said virus.
